(19) United States
(12) Patent Application Publication
(10) Pub. No.: US 2010/0010471 A1
(43) Pub. Date: Jan. 14, 2010

(54) MEDICATION APPLICATOR DEVICE

(75) Inventors: Lori A. Ladd, Chesterfield, MO (US); Lisa C. Miller, Rowayton, CT (US)

Correspondence Address: KELLEY DRYE & WARREN LLP, 400 ALTLANTIC STREET , 13TH FLOOR, STAMFORD, CT, 06901, US

(73) Assignee: Euro-Celtique S.A.

(21) Appl. No.: 12/373,933

(22) PCT Filed: Jul. 9, 2007
(86) PCT No.: PCT/EP2007/006060

§ 371 Date: Aug. 25, 2009

Related U.S. Application Data
(60) Provisional application No. 60/807,525, filed on Jul. 17, 2006.
(60) Provisional application No. 60/823,838, filed on Aug. 29, 2006.

(51) Int. Cl. A61M 31/00

(52) U.S. Cl. 604/514; 604/60

(57) ABSTRACT

A device for inserting medication into a cavity of a body comprising a syringe having a barrel member and a plunger, the plunger having an end for movable interaction with the barrel member, the end comprising a seat configured to allow seating of the medication therein.

### FIELD OF THE INVENTION

The present invention generally relates to a device for inserting medication into a cavity of a body. In a specific embodiment, there is provided a suppository inserter, and method of insertion of a suppository into a body cavity, which avoids the need for digital insertion.

### DESCRIPTION OF THE RELATED ART

Medications may be dispensed to a patient in various ways, for example, by inhalation, oral ingestion, transdermally and insertion into a body cavity. Cavity administration may be necessary for treatment, when other routes of administration, such as oral, intramuscular or intranasal are not appropriate (e.g. due to the physical state of the patient) or are not optimal (e.g., when the pharmacokinetic profile of the drug by such alternative routes is not therapeutically desirable).

Administration of medications into certain cavities of the body, such as the rectum and vagina, can be difficult and unpleasant for some patients and some caregivers. Cavity administration may entail the need for the administrator to wash his or her hands thoroughly or to use sterilized hand gloves. Administration of medications into body cavities also entails the need of the administrator to take significant care not to scratch or injure one or more walls of the cavity upon insertion, as well as the need for the administrator to take care to insert the medicament to the proper depth in the cavity. Emotional obstacles to such administration may result in drug treatment compliance failures.

Various types of devices have been proposed for administering medications into body cavities (in particular medications formulated into suppositories), including the rectum, vagina, ear, nasal cavity, and mouth (see, e.g. U.S. Pat. Nos. 2,754,822, 3,667,465, 3,934,584, 4,361,150, 5,201,779, 5,404,870, 5,860,946, and U.S. Patent Application Publication No. 2003/0233078). Applicators for administration of medications into body cavities often incorporate a channel and a plunger, with the plunger being telescopingly slidable within the channel to advance the drug through the channel and into the cavity. These devices are typically designed to enable patients and/or caregivers to insert a drug, such as in suppository form, correctly, safely, and hygienically while at the same time minimizing the unpleasant nature of insertion. Reliability of insertion may also be improved.

Several devices proposed for administering medications, in particular medications in suppository form, into body cavities suffer from deficiencies in design. For example, some applicators fail to provide for the inexperience of users, allowing the applicator to be positioned too far into the body cavity (for example, past a sphincter) causing insertion to be uncomfortable and running the risk of trauma to the body cavity. Other applicators lack the ability to adjust the level of insertion of the applicator to a comfortable penetration depth, failing to consider that body cavities vary in size and shape as does flesh thickness about the cavities (e.g. buttocks size around the anal canal). Other applicators, e.g., certain spring-loaded applicators, fail in allowing for too much force to be applied to the drug within the applicator causing violent forces to be applied to tissue when the drug is expelled from the applicator.

A number of proposed applicators designed to overcome one or more of such shortcomings suffer from complex components and design, requiring complex assembly and steps for use. This may raise concerns in maintaining the integrity of a suppository, as well as adversely affecting the user's ability to manipulate the device correctly. Where insertion is led by the actual suppository rather than the distal end of the device, there is risk of suppository damage, for example, by premature melting, crushing, adulteration or loss of integrity. Suppository damage may place the patient at risk for unpredictable dosing. Several proposed applicators further suggest plunger tables that extend beyond the distal tip of the applicator which may result in damage to the mucosa during insertion and removal. Further, design of a number of proposed applicators may allow for misplacement of the applicator in the body cavity causing obstructed or misdirected release of medicament. Lastly, the design of some applicators does not protect against undesired exposure to secretions, and may require extensive cleansing for reusability.

Given the problems with such applicators, there is a need for an applicator allowing for easy and efficient administration of medications into body cavities, in particular into the rectum, with minimal safety concerns.

### SUMMARY OF THE INVENTION

Among the embodiments disclosed herein, there is disclosed an applicator for delivering pharmaceutical products to a body cavity, the applicator comprising: an applicator body defining a through-void, the through-void being substantially symmetrical about a defined axis, the applicator body having a first open end portion and a second open end portion, the second open end portion comprising two or more resilient petals converging forwardly, and the first open end portion comprising a flange portion extending outwardly away from the defined through-void axis; a plunger having a first plunger end, a second plunger end, and an intermediate elongated portion between the first plunger end and the second plunger end, the first plunger end of the plunger being shaped to snugly fit within the through-void of the applicator body while allowing for the plunger to be moved in the through-void between the first open end portion and the second open end of the applicator body; the first plunger end further defining a horizontal table with a generally central recess configured for insertion of a pharmaceutical product, the second end comprising a base portion of sufficient area to permit force to be applied thereto to move the plunger along the through-void axis when the plunger first end is positioned in the through-void. The second plunger end of the plunger may be dimensioned so as not to fit within the through-void. The second open end portion of the applicator body may be configured so as to be insertable into the body cavity. Optionally, the device may further comprise one or more of: demarcations on the applicator body for pre-determined relevant depths for insertion of the applicator body into the body cavity; one or more stops extending into the through-void, the one or more stops operatively limiting movement of the plunger first end along the through-void; a through-void narrowing towards the first open end portion of the applicator body; a coating along the surface of the through-void comprising at least in part by lubricant; a curved flange portion of the applicator body, which may be configured to curve toward the first open end portion of the applicator body; and a flange portion of the applicator body which includes digit rests. The digit rests may be textured to allow for more controlled interaction with a digit placed thereon.

In yet another embodiment, there is disclosed an applicator for rectal insertion of a suppository, the suppository having a lead end and an inferior end, the applicator defined by a barrel component having a bottom end and a top end and a movable plunger component in the barrel, the plunger comprising a first end which is configured to snugly fit within the barrel, and a second end, the first end comprising a table defined by a border; the applicator characterized by the first end of the movable plunger defining a table defined by a border, the table having a generally concentric seat about the central axis of the table, the concentric seat being recessed from the border of the table and configured to snugly hold the inferior end of the suppository. The second end of the plunger may be sized and shaped to be unable to fit within the shaft of the barrel component. Optionally as above, the applicator may comprise demarcations on the applicator for pre-determined relevant depths for insertion of the applicator body into the body cavity; the barrel of the applicator may narrow towards one end of the applicator body, preferably the top end; the barrel of the applicator may have a curved flange portion at its bottom end; and the flange portion of the applicator body may include digit rests.

In yet a further embodiment, there is disclosed a device for placing a suppository having a first end and a second end into a body cavity, the device comprising: an applicator body with a shaft extending from a first open end to a second open end, the shaft defined by walls of the applicator body; a plunger moveably secured to the applicator body, the plunger comprising a first end sized and shaped to snugly and/or sealingly fit within the first open end of the applicator body shaft, and a second end, wherein the plunger first end comprises a table defined by a border, the table having a seat portion recessed from the border defining the table, the seat portion dimensioned to retain at least a portion of the first or second end of the suppository. The seat portion may further be configured such that when the plunger is positioned in the applicator body the suppository does not touch the walls of the applicator body. When the seat is configured to allow seating of the medication in a manner such that the suppository does not touch the walls, there is a minimization of loss of the medication in the device. The second end of the plunger may be sized and shaped to be unable to fit within the first open end of the applicator body shaft. The second open end of the applicator body may comprise one or more petals, and may be configured for insertion into the body cavity. The first open end of the applicator body may be configured not to allow insertion into the body cavity. Optionally as above, the device may comprise demarcations on the applicator body for pre-determined relevant depths for insertion of the applicator body into the body cavity; the shaft of the applicator body may narrow towards one end of the applicator body, preferably the end which is designed to be inserted into the body cavity; the applicator body may have a curved flange portion at its first open end; and the flange portion of the applicator body may include digit rests.

In yet another embodiment, there is disclosed a device for delivering a pharmaceutical product or device into a body cavity, the device comprising: a barrel component having a proximal end including a proximal opening, and a distal end including a distal opening, the distal end of the barrel component having a plurality of petals extending therefrom in a generally axial direction, and the barrel component further having a through-passageway defined by surrounding walls of the barrel component which extends the barrel component length in a direction substantially parallel to the axial direction and in communication with the openings; a plunger component of a length corresponding substantially to the barrel component length, the plunger component having a first end that is insertable in telescoping relation to at least a portion of the through-passageway of the barrel component, and a second end, the first end comprising a table with a concentric seat portion configured to hold the base portion of pharmaceutical product or device such that the pharmaceutical product or device positioned in the seat minimally impacts the surrounding walls of the barrel component on its course through the barrel component to impact with the petals. The second end of the plunger may be configured to be of a size and shape that is not insertable into the through-passageway. Options, as above, include, without limitation, demarcations on the barrel component for pre-determined relevant depths for insertion of the barrel component into the body cavity; and the barrel component having a curved flange portion at the proximal end of the barrel component.

In another aspect of the present invention, there is disclosed an applicator for delivering pharmaceutical products into a body cavity, the applicator comprising: an applicator body defining a through-void, the through-void being substantially symmetrical about a defined axis, the applicator body having a first open end portion and a second open end portion, the first and second open end portions in communication with the through-void, the second open end portion comprising two or more resilient petals converging forwardly, and the first open end portion comprising a flange portion extending outwardly away from the defined through-void axis; a plunger having a first plunger end, a second plunger end, and an intermediate elongated portion connecting the first plunger end and the second plunger end, the first plunger end of the plunger being shaped to snugly fit within the through-void of the applicator body while allowing for the plunger to be moved in the through-void between the first open end portion and the second open end portion of the applicator body; the first plunger end further defining a horizontal table with a generally central recess configured for insertion of a pharmaceutical product, the second plunger end comprising a base portion of sufficient area to permit force to be applied thereto so as to move the plunger along the through-void axis toward the second open end portion of the applicator body when the first plunger end is positioned in the through-void. The second plunger end may be dimensioned so as to be unable to fit within the through-void. The second open end portion of the applicator body is preferably configured to be insertable into the particular body cavity. Optionally, the applicator may further comprise one or more of the following: one or more demarcations on the external surface of the applicator body indicating pre-determined appropriate depths for insertion of the applicator body into the body cavity; one or more stops extending into the through-void, the one or more stops serving to operatively limit movement of the first plunger end along the through-void toward the second open end portion of the applicator body; a narrowing of the diameter of the through-void towards the second open end portion of the applicator body serving to operatively limit movement of the first plunger end along the through-void toward the second open end portion of the applicator body; a coating along the surface of the internal walls defining through-void comprising at least in part a lubricant; the flange portion of the applicator body configured to curve inwardly toward the first open end portion of the applicator body; and one or more digit or finger rests on the flange portion of the applicator body, which digit rests may be textured to allow for more controlled interaction with a finger placed thereon.

In yet another embodiment, there is disclosed an applicator for rectal insertion of a suppository, the suppository having a lead end and an inferior end, the applicator defined by a barrel component having an open bottom end and an open top end, which open bottom end and open top end are in communication with an interior space defined within the barrel component, and a movable plunger component situated within the interior space of the barrel component the plunger component comprising a first plunger end which is configured to snugly fit into the open bottom end of the barrel component and within the interior space of the barrel component, and a second plunger end, the first plunger end comprising a table defined by a border, the table having a generally concentric seat about the center of the table, the concentric seat being recessed from the border of the table and configured to snugly hold the inferior end of the suppository. The second plunger end may be sized and shaped so as to be unable to fit into the open bottom end of the barrel component or within the interior space of the barrel component. Optionally, the applicator may further comprise one or more of the following: one or more demarcations on the external surface of the barrel component indicating one or more pre-determined appropriate depths for insertion of the applicator into the rectum; one or more stops extending into the interior space of the barrel component, the one or more stops serving to operatively limit the extent of movement of the plunger component in a direction along the length of the interior space toward the open top end of the barrel component; a narrowing of the diameter of the interior space towards the open top end of the barrel component serving to operatively limit the extent of movement of the plunger component in a direction along the length of the interior space toward the open top end of the barrel component; a coating along the inner surface of the walls defining the through-void comprising at least in part a lubricant; a flange portion attached to the barrel component and adjacent to the open bottom end of the barrel component, which may be configured to curve inwardly toward the open bottom end of the barrel component; and one or more digit or finger rests on the flange portion of the barrel component, which flange portion may further comprise digit rests, which digit rests may be textured to allow for more controlled interaction with a finger placed thereon.

In yet a further embodiment, there is disclosed a device for placing a suppository having a fist end and a second end into a body cavity, the device comprising: an applicator body containing a hollow shaft extending from a first open end to a second open end in the applicator body, the hollow shaft defined by internal walls within the applicator body; a plunger that may be moveably secured to the applicator body, the plunger comprising a first plunger end sized and shaped to snugly and/or sealingly fit within the first open end of the applicator body shaft and to move along the length of the hollow shaft, and a second plunger end, wherein the first plunger end comprises a table defined by a border, the table having a seat portion recessed from the border defining the table, and the seat portion dimensioned to retain at least a portion of the first or second end of the suppository. The seat portion and suppository may further be configured such that when the first plunger end is positioned within the hollow shaft in the applicator body with a suppository seated in the seat portion, the suppository does not substantially touch the walls of the hollow shaft. When the seat is configured to allow seating of the suppository in a manner such that the suppository does not substantially touch the walls of the hollow shaft of the applicator body, there is a minimization of loss of the substance of the suppository as it moves along the length of the hollow shaft. The second plunger end may be sized and shaped so that it is unable to fit into the first open end of the applicator body or into the hollow shaft of the applicator body. The second open end of the applicator body may comprise two or more resilient petals converging in a forward or distal direction, and may be configured for insertion into the body cavity. The first open end of the applicator body may be configured to prevent its insertion into the body cavity. Optionally, the device may further comprise one or more of the following: one or more demarcations on the external surface of the applicator body indicating one or more pre-determined appropriate depths for insertion of the applicator body into the body cavity; one or more stops extending into the hollow shaft, the one or more stops serving to operatively limit the extent of movement of the first plunger end in a direction along the length of the hollow shaft toward the second open end of the applicatory body; a narrowing of the diameter of the hollow shaft towards the second open end of the applicator body serving to operatively limit the extent of movement of the first plunger end in a direction along the length of the hollow shaft toward the second open end of the applicator body; a coating on the surface of the internal walls defining the hollow shaft comprising at least in part a lubricant; a flange portion of the applicator body, which may be configured to curve inwardly toward the first open end of the applicator body; and one or more digit or finger rests on the flange portion of the applicator body, which digit rests may be textured to allow for more controlled interaction with a finger placed thereon.

In yet another embodiment, there is disclosed an applicator device for delivering a pharmaceutical product or insertable device into a body cavity, the applicator device comprising: a barrel component having a proximal end including a proximal opening, and a distal end including a distal opening, the distal end of the barrel component having two or more petals converging in a distal, generally axial direction, and the barrel component further having a through-passageway defined within it by internal surrounding walls of the barrel component, which through-passageway extends the barrel component length in a direction substantially parallel to the axial direction and in communication with the proximal and distal openings; a plunger component of a length corresponding substantially to the barrel component length, the plunger component having a first plunger end that is insertable through the proximal opening of the barrel component and movable in telescoping relation to at least a portion of the through-passageway of the barrel component, and a second plunger end, the first end comprising a table with a concentric seat portion configured to hold the base portion of a pharmaceutical product or device such that the pharmaceutical product or device when positioned in the seat will minimally impact the surrounding walls of the barrel component on its course in a distal direction through the through-passageway of the barrel component to pass through the petals. The second plunger end may be configured to be of a size and shape that is unable to pass through the proximal opening of the barrel component into the through-passageway. Optionally, the applicator device may further comprise one or more of the following: one or more demarcations on the external surface of the barrel component indicating one or more pre-determined appropriate depths for insertion of the applicator device into the body cavity; one or more stops extending into the through-passageway, the one or more stops serving to operatively limit the extent of movement of the first plunger end in a direction along the length of the through-passageway toward the distal opening; a narrowing of the diameter of the through-passageway along the length of the through-passageway toward the distal opening serving to operatively limit the extent of movement of the first plunger end toward the distal opening; a coating on the surface of the internal walls defining the through-passageway comprising at least in part a lubricant; a flange portion of the barrel component, which may be configured to curve inwardly toward the proximal end of the barrel component; and one or more digit or finger rests on the flange portion, which digit rests may be textured to allow for more controlled interaction with a finger placed thereon.

In another embodiment, there is disclosed a method for inserting a suppository into a body cavity comprising obtaining a suppository having a lead end and an inferior end; obtaining an applicator or device as defined above; appropriately loading the suppository into the applicator or device; inserting the applicator or device into the body cavity; and expelling the suppository from the applicator or device so that it is inserted into the body cavity.

In particular, the present invention further relates to a device for delivering pharmaceutical products to a body cavity, said device comprising: an applicator body defining a through-void, said through-void being substantially symmetrical about a defined through-void axis, said applicator body having a first open end portion and a second open end portion, and said first open end portion comprising a flange portion extending outwardly away from said defined through-void axis; a plunger having a first plunger end, a second plunger end, and an intermediate elongated portion connecting said first plunger end and said second plunger end, said first plunger end of said plunger being shaped to snugly fit within said through-void of said applicator body while allowing for said plunger to be moved in the through-void between said first open end portion and said second open end portion of said applicator body; said first plunger end further defining a horizontal table with a generally central recess configured for insertion of a pharmaceutical product, said second plunger end comprising a base portion of sufficient area to permit force to be applied thereto to move said plunger along said through-void axis toward said second open end portion when the plunger first end is positioned in the through-void.

The second plunger end of said plunger may be dimensioned so as not to fit within said through-void. Furthermore, the second open end portion of said applicator body may be configured so as to be insertable into the body cavity. The device may further comprise demarcations on the exterior of said applicator body demarcating pre-determined appropriate depths for insertion of said applicator body into said body cavity. In addition, the device may further comprise a means for stopping the movement of said plunger first end along said through-void as the plunger moves toward said second open end portion. In some embodiments, the means for stopping movement of said plunger is a narrowing of the diameter of the through-void toward said second open end portion of said applicator body. The applicator may further comprise one or more stops extending into said through-void defined by said applicator body. The through-void defined by said applicator body may be coated along the surface defining the through-void at least in part by lubricant. Moreover, the flange portion of said applicator body may be curved inwardly toward said first open end portion of said applicator body. The flange portion of said applicator body may include digit rests.

The present invention further particularly discloses an applicator for insertion of a suppository into a body cavity, said suppository having a lead end and an inferior end, the applicator defined by a barrel component having an open bottom end and an open top end and a shaft extending from said open bottom end to said open top end and a movable plunger component in said barrel component, said movable plunger component comprising a first end which is configured to snuggly fit within said barrel component and a second end, said first end comprising a table defined by a border; said first end of said movable plunger component having a generally concentric seat about the center of said table, said concentric seat being recessed from said border of said table and configured to snugly hold said inferior end of said suppository.

The second end of said movable plunger component may be sized and shaped to be unable to fit within said shaft of said barrel component. The applicator may further comprise demarcations on the external surface of said applicator indicating pre-determined appropriate depths for insertion of said applicator body into said body cavity. In some embodiments, the barrel narrows towards the open top end of said applicator body. The barrel component may have a curved flange portion at its open bottom end. Furthermore, the flange portion of said applicator body may include digit rests.

The present invention also specifically discloses a device for placing a suppository having a first end and a second end into a body cavity, said device comprising: an applicator body with a shaft extending from a first open end to a second open end, said shaft defined by walls of said applicator body; a movable plunger component secured to said applicator body, said movable plunger component comprising a first end sized and shaped to snuggly fit within said first open end of said applicator body shaft, and a second end sized and shaped to be unable to fit within said applicator body shaft, wherein the first end of said movable plunger component comprises a table defined by a border, said table having a seat portion recessed from said border defining said table, said seat portion dimensioned to retain at least a portion of said first or second end of said suppository such that when said movable plunger component is positioned in said applicator body, said suppository does not touch said walls of said applicator body.

In some embodiments, the second end of said plunger is sized and shaped to be unable to fit within said first open end of the applicator body shaft. In addition, the second open end of said applicator body may comprise one or more petals. The second open end of said applicator body may be configured for insertion into the body cavity. The first open end of the applicator body may be configured not to allow insertion into the body cavity. The device may further comprise demarcations on the external surface of said applicator body indicating pre-determined relevant depths for insertion of said applicator body into said body cavity. In some embodiments, the shaft of said applicator body narrows towards said second open end of said applicator body. In addition, the applicator body may have a curved flange portion at said first open end of said applicator body. The flange portion of said applicator body may include digit rests.

In addition, the present invention specifically relates to a device for delivering a pharmaceutical product or device into a body cavity, said device comprising: a barrel component having a proximal end including a proximal opening, and a distal end including a distal opening, said distal end of the barrel component having a plurality of petals converging forwardly in a generally axial direction, said barrel component further having a through-passageway defined by surrounding walls of said barrel component, which through-passageway extends said barrel component length in a direction substantially parallel to said axial direction and in communication with said openings; a plunger component of a length corresponding substantially to said barrel component length, said plunger component having a first end that is insertable through said primary opening of said barrel component and movable in telescoping relation to at least a portion of said through-passageway of said barrel component, and a second end, said first end comprising a table with a concentric seat portion configured to hold the base portion of a pharmaceutical product or device such that the pharmaceutical product or device positioned in said seat minimally impacts the surrounding walls of said barrel component on its course in a distal direction through said barrel component to pass through said petals.

The second end of said plunger is configured to be of a size and shape that is not insertable into the through-passageway. The device may further comprise demarcations on the external surface of said barrel member indicating pre-determined relevant depths for insertion of said applicator body into said body cavity. Moreover, the barrel member may have a curved flange portion at its proximal end.

The present invention further particularly discloses a method for inserting a suppository into a body cavity, the method comprising: obtaining a suppository having a lead end and an inferior end; obtaining an applicator defined by a barrel component having a bottom end and a top end and a movable plunger component in said barrel, said movable plunger comprising a first end which is configured to snuggly fit within said barrel and a second end, said first end comprising a table defined by a border; said applicator characterized by the first end of said movable plunger having a generally concentric seat about the central axis of said table, said concentric seat being recessed from said border of said table and configured to snugly hold said inferior end of said suppository; loading said inferior end of said suppository into said concentric seat of said movable plunger; inserting said plunger table into said barrel component of said applicator; and expelling said suppository from said barrel component by movement of said movable plunger within said barrel.

The present invention also specifically discloses a method for preparing a suppository applicator device defined by a barrel component having a bottom end and a top end and a movable plunger component in the barrel, the movable plunger comprising a first end which is configured to snuggly fit within the barrel and a second end, the first end comprising a table defined by a border; the applicator characterized by the first end of the movable plunger having a generally concentric seat about the central axis of the table, the concentric seat being recessed from the border of said table and configured to snugly hold an end of a suppository, said method comprising: obtaining a suppository; and inserting said suppository into said concentric seat of said first end of said movable plunger.

### BRIEF DESCRIPTION OF THE DRAWINGS

Other features and advantages of the present invention will become apparent in the following detailed description of embodiments of the invention with reference to the accompanying drawings, of which:

FIG. 1 is an isometric elevational view of the components of a suppository applicator embodiment of the present invention.

FIG. 2 is an illustration of an assembled suppository applicator embodiment of the present invention.

### DETAILED DESCRIPTION OF THE INVENTION

In embodiments described herein, there is disclosed a device (in some embodiments referred to, e.g., as applicator device) configured to permit the insertion and advancement of medications through the device and into the exterior surrounding the device. The device may be adapted for use in depositing medications, in particular in suppository form, into a body cavity, such as the rectum, vagina, etc.

In one embodiment there is disclosed a suppository applicator which includes a barrel member or component, and a plunger member or component, extending into the barrel member. The barrel member may have unitary or non-unitary construction. The barrel member defines an annular wall bounding a passageway. The barrel member may be defined by a proximal section having a structure configured to conform with the tissue surrounding the body cavity, a distal section structured for insertion into the body cavity, and an intermediate section between the proximal and distal section. The proximal section of the barrel member in cross-section may be wider than the distal section of the barrel member, and structured so as to provide a portion against which manual force can be applied, such as, for example, a flange portion extending away from the center line of the passageway. The distal section of the barrel member may be significantly narrower in cross-section than the proximal section of the barrel member to facilitate insertion of the distal section of the barrel member into the body cavity. The distal section of the barrel member may comprise no petals, or one two or more petals of material surrounding the passageway. The intermediate section may contain on its external surface an insertion depth indicator, to demarcate the depth to which the barrel member is recommended to be inserted into the specified body cavity for the particular patient (taking into account, for example, age, height, weight, etc.). The intermediate section may taper in diameter as it proceeds from the proximal section toward the distal section.

The plunger member may have a unitary or non-unitary construction. The plunger member may be divided into three major portions: a plunger table (e.g., at a plunger first end), a plunger base (e.g., at a plunger second end), and a plunger intermediate section positioned between and connecting the plunger table and plunger base. The plunger base may comprise a surface area configured to permit easy application of force to the plunger by one or more fingers. The plunger table may comprise a surface configured to fit snugly within the barrel passageway and to have a seat area thereon. The seat area may be configured to firmly or snugly hold medication by enclosing a least a portion of the medication. The medication will typically be in the form of a suppository. The seat may comprise, without limitation, a recess, which is a well or an indented area in the plunger table and/or may comprise an area bounded by walls extending upwardly from the plunger table. The plunger intermediate section may comprise an elongate member, such as a cylinder or other elongate hollow or solid member, which is fixedly attached to the plunger table at the first plunger end, and to the plunger base at the plunger second end. Attachment of the plunger table and plunger base to the plunger intermediate section may be by any fixation method, such as a polymeric melt, a polymeric adhesive, and/or a screw or socket configuration. The plunger member with its three major portions may alternatively be molded as a single unitary piece.

The barrel member and plunger member may be made from suitable materials including, without limitation, thermoplastics, polyolefins, polyolefin copolymers, modified polyvinyl chloride, thermoplastic rubber compounds, polyurethanes etc. using any number of molding processes, such as injection molding. The materials may be selected to be sterilizable, with sterilization having a minimal impact upon the physical properties of the material.

The medication dispensed by the device may be a suppository. Suppositories may comprise medication fashioned into solid bodies of various sizes and shapes suitable for introduction into body cavities. The medication is typically incorporated into a base which dissolves or melts within the body cavity in which it is placed, for example, such as cocoa butter which melts at body temperature, or glycerinated gelatin or PEG which slowly dissolves in the mucous secretions. Suppositories may be fabricated using medications that are suited for producing local action, or for producing a systemic effect, or for exerting a mechanical effect (e.g., to facilitate emptying the lower bowel). Suppository base compositions may serve an important role in both the rate and extent of release of the medication. Suppositories may comprise oleaginous (fatty) bases or water soluble or miscible bases. Oleaginous bases include theobroma oil and synthetic triglyceride mixtures, which are solid at ordinary room temperatures of about 15° to about 25° C. (59° to 77° F.), but which melt to a bland, nonirritating oil at about 30° to about 35° C. (86° to 95° F.), i.e., at body temperature. Oleaginous bases may also include synthetic triglycerides that consist of hydrogenated vegetable oils. Water soluble/water miscible bases include those containing glycerinated gelatin or the polyethylene glycol (PEG) polymers.

Suppositories may be selected from a variety of shapes, sizes and compositions based upon the body cavity into which they are to be inserted and the condition to be treated.

For example, without limitation, a two gram suppository may be approximately 1.27 cm in diameter. For such exemplar suppository, in the case of an applicator embodiment having a plunger component comprising a table having a seat, the seat may need to be slightly larger, on the order of about 1.3-1.4 cm, with the table accommodating the seat being yet slightly larger, and the shaft of the applicator allowing accommodation of such plunger component table.

An applicator may comprise one or more visible demarcations on the external surface of the barrel component to indicate the depth to which the applicator barrel is recommended to be inserted into the body cavity for desired insertion depth of the medicament into the body cavity. For example, the demarcations may be placed on the surface of a rectal suppository insertion device. Given that the average anal canal may be about 2 to about 4 cm in length, and the suppository may need to be inserted to or beyond this point, the insertion depth indicator may be placed, for example, about 4 cm back from the distal end of the applicator.

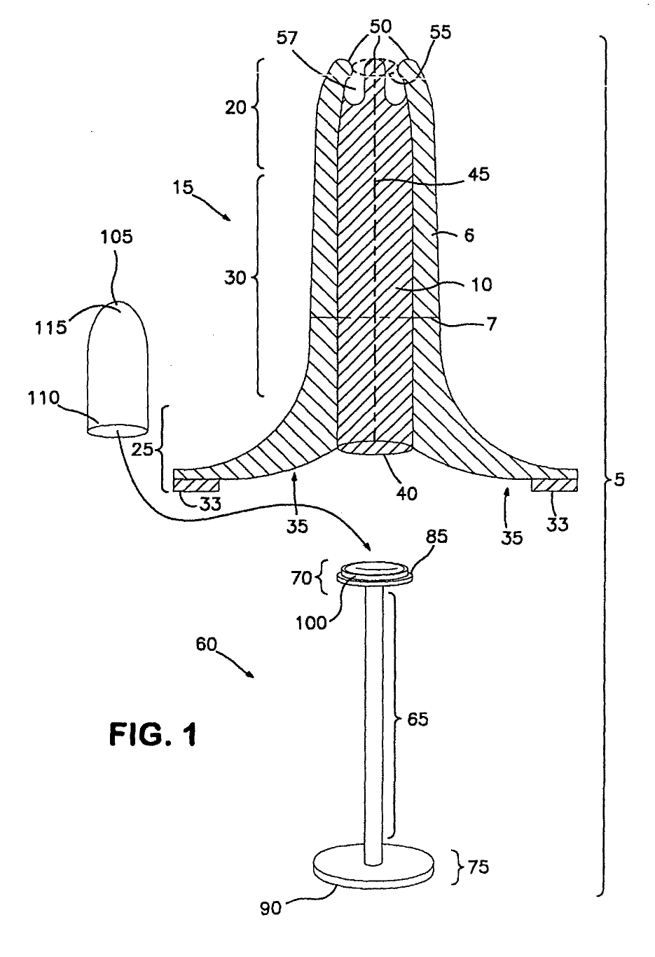

Referring to the drawing, FIG. 1 is an isometric elevational view of the components of applicator 5 designed to dispense suppositories. Applicator barrel 15 is generally in the form of a tube of unitary construction enclosing an interior through-passageway 10, defined by the annular walls 6 of applicator barrel 15, through-passageway 10 extending through applicator barrel 15. Applicator barrel 15 may be divided into a top or distal portion 20, a bottom or proximal portion 25, and an intermediate portion 30 there between. In this embodiment, bottom/proximal portion 25 is equipped with a finger push and grip flange area 35 (which may be ribbed), which includes digit rest 33, and which flanks open bottom end 40. As illustrated, in this embodiment, bottom/proximal portion 25 of applicator barrel 15 has a wider base area than top or distal portion 20, and flares outwardly from imaginary central axis 45 within through-passageway 10. Top/distal portion 20 is configured to fit within the body cavity into which suppository 105 is to be placed, that is, it is configured to function as an insertion tip. Top/distal portion 20 is shown as comprising a number of flexible, truncated petals 50 encircling head opening 55. Each petal 50 is connected to top/distal portion 20 at its base and extends generally at its top inwardly towards imaginary central axis 45 within through-passageway 10. Although not shown, petals 50 may be constructed to extend higher towards the central axis of the opening for dispensing the suppository. For example, petals 50 may be configured such that they almost meet one another at their most distal point. Petals 50 cooperate with top/distal opening 55 so as to form a receptacle 57 for releasably receiving a suppository therein. Each of petals 50 are shown to have an axial length sufficient to hold a suppository within the receptacle 57 until the suppository advances through-passageway 10 to an extent that it is to be released into the body cavity, but wherein the axial length of the petals is short enough to create a minimal frictional resistance to the suppository during its dispensing into the body cavity. Applicator barrel 15 may comprise one or more demarcations 7 thereon/therein indicating the insertion distance calculated to be advantageous in respect of applicator barrel 15 into the body cavity.

Plunger 60 has a top plunger portion 70, a bottom plunger portion 75, and intermediate shaft plunger portion 65. Top plunger portion 70 is shown to comprise a plunger table 85. Bottom plunger portion 75 is shown to comprise a force platform 90. Force platform 90 is shown to comprise a disc, although other shapes and non-flat forms may be used. Force platform 90 provides an area for force, such as asserted by a finger, to push on plunger 60. Plunger table 85 is shown to comprise seat 100 which as shown is an encircling ledge on top plunger table 85, but may comprise any structure or physical feature capable of holding the suppository, such as a hollowed area, or a combination of both. Seat 100 may form a press fit with the non-leading end 110 of a suppository 105, such fit which provides enough grip to hold the suppository on plunger table 85 of plunger 60 as it is inserted into interior through-passageway 10, but not so tight as to keep the suppository from releasing from table 85 when plunger 60 is moved to a stop position (i.e., a position wherein the plunger can no longer move upon application of force to force platform 90) in interior through-passageway 10 at top/distal portion 20 of applicator barrel 15. Plunger table 85 is configured to be movably sealable within the walls of applicator barrel 15 surrounding interior through-passageway 10. Plunger 60 is movable relative to the applicator barrel 15 in the axial direction between a retracted position in which plunger table 85 is positioned adjacent to bottom barrel portion 25 and an extended position in which plunger table 85 is located adjacent to applicator barrel top/distal portion 20. Force platform 90 may be configured to be of such size as to prohibit such platform from entering into interior through-passageway 10. Plunger table 85 may be sized so as to prohibit its movement beyond a point near or before the base of petals 50 of applicator barrel 15, thus keeping plunger 60 from entering into the body cavity. Plunger table 85 may be somewhat flexible allowing for easier release of suppository 105 from seat 100 when plunger table 85 reaches a stop position within interior through-passageway 10. Intermediate shaft plunger portion 65 may be configured to be straight to permit enhanced force against plunger table 85 when pressure is applied at force platform 90.

In conjunction, applicator barrel 15 and plunger 60 are configured to cause the linear motion of plunger 60 to be impeded at a position along interior through passageway 10. Such impeding juncture may be caused, for example, by narrowing of the applicator barrel 15 to a dimension which is narrower than plunger table 85, or by one or more stops placed within the barrel and/or on plunger 60 which interact with the respective plunger/barrel.

In use of the embodiment illustrated, a suppository 105 is prepared for insertion into the seat 100 of plunger table 85. All wrappers or covers on suppository 105 are first removed before insertion of the same into seat 100. Insertion of suppository 105 into seat 100 of plunger 60 may be performed by placing any designed non-leading edge 110 (as opposed to leading edge 115) of the suppository (e.g. the flat end) into seat 100, and then inserting the top plunger portion 70 of plunger 60 carrying suppository 105 through open bottom end 40 into interior through-passageway 10 of the bottom portion 25 of applicator barrel 15. Seat 100 therefore acts not only to push the suppository but also to hold and position it on plunger table 85. Seat 100 may also be positioned to keep suppository 105 from touching the interior walls defining interior through-passageway 10. Plunger 60 is pushed via force platform 90 to move plunger table 85 to a stop position below petals 50 thus stopping further movement of plunger table 85, such stop comprising, for example, a narrowing of the barrel to a diameter less than that of plunger table 85, or one or more physical stops jutting into the interior through-passageway 10. The stop or stops may be located so as to allow the leading edge 115 of suppository 105 to be positioned with respect to the tips of petals 50 and receptacle 57 such that the suppository 105 is dislodged or can be dislodged from suppository applicator 5 when suppository applicator 5 is in position in the body cavity or is removed from the body cavity.

Suppository applicator 5 can be provided to a user without the suppository 105 pre-loaded, or can be provided to a user with the suppository 105 pre-loaded. When provided in its pre-loaded form, suppository applicator 5 may be packaged in a blister packaging assembly. Seat 100 of plunger table 85 is structurally configured to ensure proper dispensing of suppository 105 from receptacle 57. Seat 100 may further be configured to help minimize distortion of suppository 105 as it moves through the interior of applicator barrel 15 through through-passageway 10 by minimizing contact of suppository 105 with the interior walls surrounding through-passageway 10.

In one embodiment, there is disclosed a suppository applicator 5 which is disposable. Such disposable suppository applicator may be made of one or more biodegradable materials, for example, starch impregnated polymers. Suppository applicator 5, or certain components of suppository applicator 5, such as applicator barrel 15 may comprise pliable material allowing for an appropriate degree of flexibility when inserted into the body cavity. In addition, table 85 and/or seat 70 of plunger 60 may be constructed of flexible material to aid in release of suppository 105 when table 85 reaches the stop position in interior through-passageway 10.

Suppository applicator 5 may have numerous modifications and variations. For example, the suppository applicator may carry medications in other than suppository form, or other medical objects, with the applicator being modified to dispense the medication/objects into a body cavity. Top/distal portion 20 of applicator barrel 15 can include any number of petals 50, including none (e.g., only a tapered head), one, two, three, four, five, or more petals, and such petals 50 may be provided with varying shapes and lengths. Suppository applicator 5 while useful in administering suppositories into the rectum, can alternatively be used to dispense suppositories, or other pharmaceutical products, into other body cavities such as the vagina. Suppository applicator 5 may also include a lubricating substance, which may be pre-disposed on the exterior of the applicator, such as at top/distal portion 20 of applicator barrel 15, to ease insertion of the applicator into the body cavity, and/or located along the walls of interior through-passageway 10 to ease movement of suppository 105 (and/or plunger 60) through interior through-passageway 10 of applicator barrel 15. Top/distal portion 20 of applicator barrel 15 may be configured to allow fit with a cover cap, which cover cap may be employed to help keep foreign matter from gaining entrance into interior through-passageway 10 prior to use of suppository applicator 5.

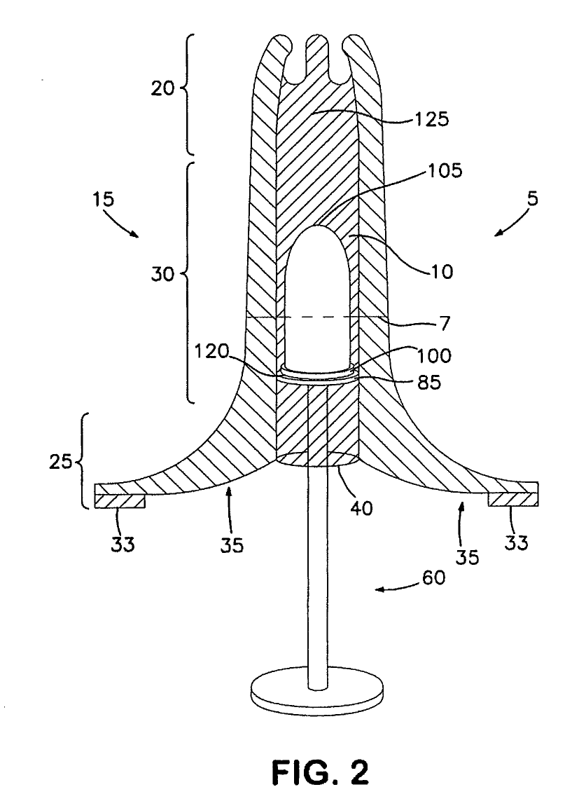

Now turning to FIG. 2, there is shown the assembly of the components of FIG. 1 to form an assembled suppository applicator 5. As shown, plunger 60 can be moved between a first position 120 in interior through-passageway 10 of applicator barrel 15 to a second position 125 in interior through passage 10. As shown, second position 125 is a terminal position in the course of plunger 60 towards top/distal portion 20 of applicator barrel 15. Such terminal position is shown to be caused by a narrowing of interior through-passageway 10 which acts as a stop for plunger 60 due to the inability of plunger table 85 to pass such point. Applicator barrel 15 may include one or more demarcations 7 on the outer surface of applicator barrel 15 to demarcate one or more desirable insertion depths for applicator barrel 15 into the particular body cavity. Movement of plunger 60 in applicator barrel 15 can be discerned if the material comprising applicator barrel 15 is translucent, or semi-translucent.

Suppository applicator 5 may be provided along with a disposable sheath that is sized and shaped to fit over top/distal portion 20 of applicator barrel 15 typically at least to one or more demarcations 7. By use of disposable sheaths, suppository assembly 5 may not need to be disposed each time a suppository is inserted into a body cavity (rather the sheath is discarded and suppository applicator 5 used again with another sheath). Sheaths may be composed of any material useful for this purpose, including a biodegradable material. In an embodiment, the sheaths and/or suppository applicator 5 may be discarded by first placing them in a disposal bag or other disposal container, which may be preferably opaque, and which may be scented or comprise an odor-absorbing material, and then discarded into a trash receptacle.

Referring generally to both FIG. 1 and FIG. 2, in application, a suppository is placed into seat 100 of plunger 60 and then inserted into interior through-passageway 10 of applicator barrel 15 through opening 40. Top/distal portion 20 of suppository applicator 5 is then inserted into the body cavity, for example, the rectum. Plunger 60 is then depressed to move plunger table 85 along interior through-passageway 10 toward top/distal portion 20 until a stop position is reached and/or the suppository enters the body cavity. Suppository applicator 5 is then removed by pulling back on barrel grip flange area 35.

### Statement Regarding Preferred Embodiments

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

1. A device for delivering pharmaceutical products to a body cavity, said device comprising:
an applicator body defining a through-void, said through-void being substantially symmetrical about a defined through-void axis, said applicator body having a first open end portion and a second open end portion, and said first open end portion having extended therefrom and outwardly away from said defined through-void axis, a flange portion;
a plunger having a first plunger end, a second plunger end, and an intermediate elongated portion connecting said first plunger end and said second plunger end, said first plunger end of said plunger being shaped to snugly fit within said through-void of said applicator body while allowing for said plunger to be moved in the through-void between said first open end portion and said second open end of said applicator body; said first plunger end further defining a horizontal table with a generally central area configured for insertion of a pharmaceutical product, said second plunger end comprising a base portion of sufficient area to permit force to be applied thereto to move said plunger along said through-void axis toward said second open end portion when the plunger first end is positioned in the through-void.

2. The device of claim 1, wherein said second plunger end of said plunger is dimensioned so as not to fit within said through-void.

3. The device of claim 1, wherein the second open end portion of said applicator body is configured so as to be insertable into the body cavity.

4. The device of claim 1, further comprising demarcations on said applicator body demarcating pre-determined appropriate depths for insertion of said applicator body into said body cavity.

5. The device of claim 1, further comprising means for stopping movement of said plunger first end along said through-void as the plunger moves toward said second open end portion.

6. The device of claim 5, wherein said means for stopping movement of said plunger is narrowing of the diameter of the through-void in the direction of the second open end portion of said applicator body.

7. The device of claim 5, wherein said means for stopping movement of said plunger is one or more stops extending into said through-void.

8. The device of claim 1 wherein the walls defining said through-void are coated along their surface at least in part by lubricant.

9. The device of claim 1 wherein said flange portion of said applicator body is curved inwardly toward said first open end portion of said applicator body.

10. The device of claim 1 wherein said flange portion of said applicator body includes digit rests.

11. The device of claim 1, wherein said generally central area of said horizontal table of said first plunger end comprises an encircling wall extending from said horizontal table.

12. The device of claim 1, wherein said applicator body is fabricated from a pliable biodegradable material.

13. The device of claim 1, wherein said-pliable degradable material is a starch impregnated polymer.

14. An applicator for insertion of a suppository having a lead end and an inferior end into a body cavity, the applicator comprising: a barrel component having an open bottom end and an open top end, and a concentric shaft defined by an internal barrel wall running from said open bottom end to said open top end, said internal barrel wall including one or more stops protruding therefrom into said concentric shaft; and a movable plunger component comprising a first end which is positioned in said concentric shaft of said barrel component and is configured to snuggly fit within said shaft, and a second end which is configured so as not to be able to fit within said shaft of said barrel component, said first end of said movable plunger comprising a table having a generally concentric seat about the center of said table configured to snugly hold said inferior end of said suppository.

15. The applicator of claim 14, further comprising demarcations on the external surface of said applicator indicating predetermined appropriate depths for insertion of said applicator body into said body cavity.

16. The applicator of claim 14, wherein said barrel component narrows toward the open top end of said applicator body.

17. The applicator of claim 14, wherein said barrel component has a curved flange portion at its open bottom end.

18. The applicator of claim 17, wherein said flange portion of said applicator body includes digit rests.

19. The applicator of claim 14, wherein said generally concentric seat of said table of said first plunger end comprises an encircling wall extending from said horizontal table.

20. The applicator of claim 14, wherein said applicator body is fabricated from a pliable biodegradable material.

21. A device for placing a suppository having a first end and a second end into a body cavity, said device comprising:
an applicator body having a first open end and a second open end with a shaft extending from said first open end to said second open end, said shaft being defined by walls of said applicator body;
a flange portion extended outwardly from said first open end of said applicator body, said flange portion including at least two parallel digit rest areas which are distinguishable from the remainder of said flange portion;
a movable plunger component inserted in said applicator body, said movable plunger component comprising a first end sized and shaped to snuggly fit within said first open end of said applicator body shaft, and a second end sized and shaped to be unable to fit within said applicator body shaft, wherein the first end of said movable plunger component comprises a table defined by a border, said table having a seat portion recessed from said border defining said table, said seat portion dimensioned to retain at least a portion of said first or second end of said suppository such that when said movable plunger component is positioned in said applicator body, said suppository does not touch said walls of said applicator body.

22. The device of claim 21, wherein said second open end of said applicator body comprises one or more petals.

23. The device of claim 21, wherein said second open end of said applicator body is configured for insertion into the body cavity.

24. The device of claim 21, further comprising demarcations on the external surface of said applicator body indicating pre-determined relevant depths for insertion of said applicator body into said body cavity.

25. The device of claim 21, wherein said shaft of said applicator body narrows toward said second open end of said applicator body.

26. The device of claim 21, wherein said flange portion is curved toward said second open end of said applicator body.

27. The device of claim 21, wherein said seat portion recess of said table of said first plunger end comprises an encircling wall extending from said horizontal table.

28. The device of claim 21, wherein said applicator body is fabricated from a pliable biodegradable material.

29. A device for delivering a pharmaceutical product or device into a body cavity, said device comprising:
a barrel component having a proximal end having a proximal opening, and a distal end having a distal opening, said barrel component further having a generally straight and uniform through-passageway extending from said proximal opening to said distal opening, said through-passageway defined by surrounding walls of said barrel component; said proximal opening having extended therefrom, and outwardly away from said through-passageway, a flange portion having digit rests incorporated thereon;
a plunger component of a length corresponding substantially to said barrel component length, said plunger component having a first end that is insertable through said proximal opening of said barrel component and movable in telescoping relation to at least a portion of said through-passageway of said barrel component, and a second end, wherein said first end comprises a table with a concentric seat portion configured to hold the base portion of a pharmaceutical product or device such that the pharmaceutical product or device positioned in said seat minimally impacts the surrounding walls of said barrel component on its course in a distal direction through said barrel component.

30. The device of claim 29 wherein said second end of said plunger component is configured to be of a size and shape that is not insertable into the through-passageway.

31. The device of claim 29, further comprising demarcations on the external surface of said barrel component indicating pre-determined relevant depths for insertion of said applicator body into said body cavity.

32. The device of claim 29, wherein said barrel component has one or more protrusions extending from the wall defining the through-passageway into the through-passageway, which one or more protrusions serve to stop the further movement of said plunger component toward said distal end.

33. The device of claim 29, wherein said concentric seat portion of said table of said first plunger component end comprises a encircling wall extending from said horizontal table.

34. The device of claim 29, wherein said barrel component is fabricated from a pliable biodegradable material.

35. A method for inserting a suppository into a body cavity:
obtaining a suppository having a lead end and an inferior end;
obtaining an applicator comprising: a barrel component having a bottom end having at least one outwardly extending flange portion comprising at least a pair of digit rests, and a top end; and a movable plunger component having a first end which is configured to snuggly fit within said barrel and a second end, said first end comprising a table defined by a border and having a generally concentric seat about the central axis of said table, said concentric seat being recessed from said border of said table and configured to snugly hold said inferior end of said suppository;
loading said inferior end of said suppository into said concentric seat of said movable plunger component;
inserting said plunger table carrying said loaded suppository into said barrel component of said applicator;
inserting said top end of said barrel component into the orifice of said body cavity while pressing on said digit rests of said at least one flange; and
expelling said suppository from said barrel component by pushing said movable plunger within said barrel toward said top end.

36. The method of claim 35, further comprising the step of placing a disposable sheath of material over said top end of said barrel component prior to inserting said top end of said barrel component into said orifice.

37. The method of claim 35, further comprising removing said applicator from said orifice by grabbing said flange portion and pulling said applicator from said orifice after expelling said suppository from said barrel component.
